(19) United States
(12) Patent Application Publication
(10) Pub. No.: US 2004/0265288 A1
(43) Pub. Date: Dec. 30, 2004

(54) New applications of gene therapy technology

(76) Inventor: Michael Z. Gilman, Newton, MA (US)

Correspondence Address: ROPES & GRAY LLP, ONE INTERNATIONAL PLACE, BOSTON, MA, 02110-2624, US

(21) Appl. No.: 10/893,199

(22) Filed: Jul. 16, 2004

Related U.S. Application Data
(63) Continuation of application No. 09/676,834, filed on Sep. 29, 2000.
Application No. 09/676,834 is a continuation of application No. 09/151,001, filed on Sep. 10, 1998, now abandoned.
Application No. 09/151,001 is a continuation-in-part of application No. 08/813,771, filed on Mar. 7, 1997, now abandoned.
Application No. 08/813,771 is a continuation-in-part of application No. 08/400,643, filed on Mar. 8, 1995, now abandoned.
(60) Provisional application No. 60/013,014, filed on Mar. 7, 1996.

(30) Foreign Application Priority Data
Mar. 9, 1998 (WO) PCT/US98/04525

(51) Int. Cl. A61K 48/00; C12N 5/08

(52) U.S. Cl. 424/93.21; 514/44; 435/366

(57) ABSTRACT

This invention provides new applications of regulated gene therapy relating to treatment of cancer and other disorders.

This application claims the priority benefit of PCT US98/04525, filed Mar. 9, 1998, and is a continuation-in-part of 08/813,771, filed 317/97, which is a continuation-in-part of 08/400,643, filed Mar. 8, 1995, now abandoned, and of provisional application 60/013,014, filed Mar. 7, 1996.

### INTRODUCTION

Regulatable transcription of introduced genes in response to administered pharmaceutical agents represent a significant new development in gene therapy. Notable among those systems are the dimerization-controlled system of Schreiber et al (see e.g. U.S. Ser. No. 08/017,931 (filed Feb. 12, 1993) and modifications thereof (see e.g. U.S. Ser. No. 08/292,598, filed Aug. 18, 1994 and PCT/US95/10559, filed Aug. 18, 1995), Gilman U.S. Ser. No. 08/292,599 (filed Aug. 18, 1994), Gilman U.S. Ser. No. 08/373,351 (filed Jan. 17, 1995) and Holt U.S. Ser. No. 60/015,502 (filed Feb. 9, 1996).

The development of regulated transcriptional systems now permits new applications of gene therapy.

### SUMMARY OF THE INVENTION

This invention provides, among other aspects, engineered cells which, following exposure to a selected ligand, express a target gene encoding an angiogenesis inhibitor protein, a tumor-specific antigen such as the carcinoembryonic antigen (CEA), a cytokine or (in the case of non-cutaneous cells) beta interferon. Angiogenesis inhibitors include, among others, thrombospondin, angiostatin, endostatin, angiostatin-endostatin fusion proteins, angiopoietin, a soluble receptor for VEGF (e.g. Flk-1 or Flt-1), a dominant negative form of VEGF, anti-VEGF antibodies, soluble Tie2/Tek receptor, and the 16 kD fragment of prolactin and related members of the prolactin-growth hormone-placental lactogen family, e.g. proliferin related protein and placental lactogen II.

These cells contain a first DNA construct (or pair of such constructs) encoding chimeric protein molecules comprising (i) at least one receptor domain capable of binding to a selected ligand and (ii) another protein domain, heterologous with respect to the receptor domain, referred to as the “action” domain. The chimeric proteins, either alone or in combination with additional chimeric proteins, are capable of triggering the activation of transcription of a target gene. The target gene in these cells is under the transcriptional control of a transcriptional control element responsive to binding of ligand to the ligand binding domain. These cells further contain a target gene whose expression is responsive to the binding of ligand to the ligand binding domain, i.e. to the presence of ligand. In the regulated expression system of Schreiber et al (see U.S. Ser. No. 08/017,931) the chimeric proteins multimerize upon addition of ligand and transcription of the target gene is responsive to the multimerization of the chimeric proteins. However, alternative embodiments comprise ligand-mediated systems such as those regulated by tetracycline, RU486 or ecdysone. These systems are more fully described in Natesan et. al., U.S. Ser. No. 09/126,009, filed Jul. 29, 1998, (Atty Docket number 363B). In certain embodiments where an alternative ligand-mediated system is used, the chimeric protein consists essentially of one or more ligand binding domains, a DNA binding domain and a transcription activation domain which is heterologous with respect to the ligand binding domain. Any ligand binding domain may be used in the design of chimeric proteins for the practice of this invention. Exemplary ligand binding domains include immunophilins, cyclophlins, steroid hormone binding domains and antibiotic binding domains. In most cases, the target gene preferably encodes a peptide sequence of human origin.

This invention thus provides a method for rendering cells capable of regulatable expression of one of the target genes disclosed herein following exposure of the cells to a selected ligand. In this method one introduces into the cells DNA constructs encoding the chimeric protein molecules and a target DNA construct. These constructs may be introduced into cells removed from a human or non-human animal or other organism and maintained in vitro. Thus, cells can be engineered ex vivo and subsequently introduced into the recipient. Alternatively, the constructs may be introduced into cells in situ within a host organism by administration of the DNA directly to the organism. In one embodiment, the constructs are delivered via viral vectors, e.g., adenovirus, adeno-associated virus, herpesvirus or retrovirus, as described below. See also Natesan et al., U.S. Ser. No. 09/126,009, supra.

Introducing constructs of this invention, or cells containing them, into a human or non-human organism, combined with administration of a ligand capable of effecting expression of the target gene provides a means for treating diseases responsive to the target gene product. Said differently, a human or non-human host organism containing cells engineered in accordance with this invention may be treated for diseases responsive to or affected by the expression of the introduced target gene. For example, in the case of cancer treatments, there have been a number of recent reports confirming the inhibition of tumor growth following treatment with angiogenesis inhibitors. See, e.g., Cao et al., J. Clin. Invest., 101:1055-1063, March 1998 (angiostatin), Millauer et al., Cancer Research 56:1615-1620, Apr. 1, 1996; Lin et al., Cell Growth and Differentiation, 9:49-58, January 1998, (soluble receptors for VEGF, Flk-1 and Flt-1), Kim et al., Nature 362:841-844, Apr. 29, 1993 (anti-VEGF antibodies). Thus, a human or non-human host organism containing such cells, where the introduced target gene is a DNA sequence encoding thrombospondin, angiostatin, endostatin, angiostatin-endostatin fusion proteins, angiopoietin, a soluble receptor for VEGF, a dominant negative form of VEGF, anti-VEGF antibodies, soluble Tie2/Tek receptor, the 16 kD fragment of prolactin, a tumor-specific antigen such as the carcinoembryonic antigen (CEA) or a cytokine (such as IL-2, IL-4, IL-7, IL-12, GM-CSF, gamma interferon, etc), may be treated for various cancers by administration of a ligand capable of mediating the transcription of those genes and the resulting production of the therapeutic protein. Where the introduced target gene encodes a beta interferon protein such as interferon β-1B, administration of the ligand constitutes a method for treating MS episodes. In the case of multiple sclerosis, direct intramuscular injection of beta-interferon is currently the treatment of choice. Such treatment reduces the frequency and/or severity of MS episodes. Thus, in the subject invention, treatment of MS would be effected via regulatable expression of the gene for beta-interferon. Where the introduced target gene encodes a ribozyme or antisense sequence directed to an RNA or DNA encoding a component required for the persistence or spread of HIV, administration of the ligand constitutes a method for treating HIV infection. Preferably the ligand is administered in a pharmaceutical composition which further contains pharmaceutically acceptable excipients. Administration may be by any of the various acceptable routes and will comprise an effective amount of the ligand to effect observable expression of the target gene, whether observed directly, e.g. by observation of the production of the target gene product, or indirectly, e.g. by observation of the effects of production of the target gene product.

With respect to applications to cancer treatment, we note the following. A promising new avenue for cancer therapy is “vaccinating” a patient against his own tumor. Two general strategies are being explored for gene therapy-directed cancer vaccines. One is to augment the immunogenicity of tumor cells by transferring into the cells an engineered gene that directs the production of cytokine that can recruit and locally activate cells of the immune system. Among the cytokines that have been used for this purpose are: interleukin-2, interleukin-4, interleukin-12, interferon-gamma, and granulocyte/macrophage colony-stimulating factor. It is contemplated that effectiveness of this approach is limited by the need to produce cytokines within an optimal therapeutic window. Enhanced cytokine production more effectively recruits and activates the immune system, but systemic circulation of these potent molecules can be toxic. Therefore, the ability to adjust cytokine production precisely and to terminate production if toxic side effects develop will be critical to the practical implementation of tumor vaccination.

In the second strategy, patients are immunized against molecularly-defined tumor-specific antigens. One example is the carcinoembryonic antigen (CEA), which is expressed by many colorectal tumors. Vaccination against molecularly-defined antigens can be achieved using simple techniques known in the art, such as intramuscular injection and microprojectile bombardment, to deliver DNA molecules directing the production and display of the antigen. Animal experiments indicate that this vaccination strategy generates both cellular and humoral immunity. However, the present inventor contemplates that the effectiveness of DNA-mediated vaccination in humans will be limited by the level of antigen expression. For DNA vaccines delivered by intramuscular injection, current technology may require that patients be injected with large amounts of DNA. The subject invention provides materials and methods with which one may reduce the amount of DNA required, preferably by tenfold or more. Furthermore, there may be an optimal level of antigen expression for eliciting an immune response, and more complex immunization schemes, such as “pulsing” the immune system with antigen, may be significantly more effective. These approaches are only possible in conjunction with regulated gene therapy. Finally, regulated antigen expression from a stably-delivered gene cassette provides an opportunity for periodic administration of “boosters.” Thus, for example, the anti-tumor activities of the immune system could be redeployed as needed to fight off recurrences of disease.

We contemplate two different preferred routes for antigen presentation. The simplest is intramuscular injection of DNA or viral vectors containing the constructs described herein. In addition, however, the use of ex vivo stem, progenitor, and/or peripheral cell gene therapy is also contemplated for delivering antigen-encoding DNA into professional antigen-presenting cells. This strategy has three significant advantages over intramuscular injection. First, these cells are specialized for antigen presentation and they circulate in the body, suggesting that the potency of antigen presentation and of the resulting immune response will be greater than with intramuscular delivery. Second, by directing expression to different types of antigen-presenting cells, the balance between cellular and humoral immunity can be controlled. Third, because transduction is stable, immunization can be life-long and modulated as needed by the administration of ligand. In various embodiments of this invention, and particularly in the case of “vaccination” via gene therapy, the chimeric responder constructs are preferably under the expression control of cell-type- or tissue-specific regulatory elements, e.g. promoters and/or enhancers. See e.g. U.S. Ser. No. 08/292,596 (filed Aug. 18, 1994)(especially page 24 et seq thereof). Thus where the engineered cells are hematopoietic stem cells or progenitors cells, use of regulatory elements from immunoglobulin heavy or light chain genes (e.g. the Ig kappa light chain promoter/enhancer) provides for B-cell-specific expression of the chimeric proteins, and thus B-cell-specific expression of the target gene. See e.g., Borelli, E. et al. (1988) Targeting of an inducible toxic phenotype in animal cells. Proc. Natl. Acad. Sci. USA 85:7572-7576; and, Heyman et al (1989) Thymidine kinase obliteration: creation of transgenic mice with controlled immunodeficiencies. Proc. Natl. Acad. Sci. USA 86:2698-2702. One can provide for macrophage-specific expression of the responder constructs by using the gp91-phox promoter in the responder constructs. See e.g., Targeting of transgene expression to monocyte/macrophages by the gp91-phox promoter and consequent histiocytic malignancies. Proc Natl Acad Sci USA 88: 8505-9 (1991). See also L. C. Burkly et al, J Immunol 142: 2081-2088 (1989) regarding B cell and macrophage-specific expression using expression control elements from the class II E alpha d gene.

Design, production and use of DNA constructs and ligands for the practice of this invention may take advantage of principles, materials and methods disclosed in any of the following: PCT/US94/01667 and PCT/US94/08008 (Crabtree and Schreiber et al), PCT/US95/16982 (Pomerantz et al), PCT/US95/10559 and PCT/US97/03137 (Holt et al), PCT/US96/09948 (Clackson et al), PCT/US95/10591 (Brugge et al) and PCT/US97/22454 (Cerasoli), as well as US priority applications identified in any of the foregoing.

### DETAILED DESCRIPTION

This invention provides for the delivery of pharmacologically active agents for the treatment and/or prevention of HIV, cancer and MS via gene therapy, preferably via regulatable gene therapy. The invention involves recombinant DNA constructs (“target gene constructs”) containing a first DNA sequence encoding a target gene and a second DNA sequence comprising a transcriptional regulatory element, such as a promoter or enhancer sequence, which is responsive to the presence of ligand, as is discussed in detail below. Target genes of this invention which are relevant to the treatment of cancer are DNA sequences encoding a cytokine such as IL-2, IL-4, IL-7, IL-12 or GM-CSF; an angiogenesis inhibitory factor such as thrombospondin, angiostatin, endosta tin, angiostatin-endostatin fusion proteins, angiopoietin, a soluble receptor for VEGF, a dominant negative form of VEGF, anti-VEGF antibodies, soluble Tie2/Tek receptor, the 16 kD fragment of prolactin and related members of the prolactin-growth hormone-placental lactoglobulin family; and a tumor-specific antigen such as the carcinoembryonic antigen (CEA). A target gene relevant to MS encodes a beta interferon protein such as IFN beta-1B. Preferably, the foregoing target genes encode a protein of human origin or sequence to avoid undue risks of antigenicity. A target gene relevant to the treatment or prevention of HIV infection is a DNA sequence comprising a ribozyme or antisense sequence directed to an RNA or DNA sequence, respectively, for an HIV component required for the reproduction or spread of the virus. See e.g. Dropulic and Jeang, 1994, Human Gene Therapy 5:927-939 (and references cited therein).

DNA sequences for target genes may be readily obtained by conventional means. For instance, primers may be designed based on the published sequence of a desired target cDNA, synthesized by conventional procedures and used in obtaining target gene DNA through standard PCR techniques. DNA sequence information and other information relevant to the cloning and use of target gene sequences are readily available, as illustrated in the following table:

| | | Genbank |
|---|---|---|
| target gene | reference | Accession # |
| IFN-beta | S. Ohno & T. Taniguchi (1981). Structure of a | J00218 |
| | chromosomal gene for human interferon beta. Proc. | |
| | Natl. Acad. Sci. U.S.A. 78, 5305-5309. | |
| | R. Crea & D. V. N. Goeddel. Polypeptides, process for | |
| | their microbial production, intermediates therefor and | |
| | compositions containing them. | |
| | EuropeanPatent 0041313-A W. C. Fiers. DNA | |
| | sequences, recombinant DNA molecules and processes | |
| | for producing human fibroblast interferon. | |
| thrombospondin | J. Lawler & R. O. Hynes (1986) J. Cell Biol. 103, 1635-1648. | X14787 |
| | S. W. Hennessy, et al (1989) J. Cell Biol. 108, 729-736. | |
| VEGF | D. W. Leung, et al (1989). Vascular endothelial growth | M32977 |
| | factor is a secreted angiogenic mitogen. Science 246, | |
| | 1306-1309. | |
| | U.S. Pat. No. 5332671 | |
| VEGF-R | B. Millauer et al, Cell 72: 835-846 (1993) | U01134 |
| CEA | S. Oikawa, H. Nakazato & G. Kosaki (1987). Primary | M17303 |
| | structure of human carcinoembryonic antigen (CEA) | |
| | deduced from cDNA sequence. Biochem. Biophys. Res. | |
| | Commun. 142, 511-518. | |
| IL-2 | Taniguchi et al (1983). Structure and expression of a | X01586 |
| | cloned cDNA for human interleukin-2. Nature 302, | |
| | 305-310. | |
| | R. Devos, et al (1983). Molecular cloning of human | |
| | interleukin 2 cDNA and its expression in E. coli. | |
| | Nucleic Acids Res. 11, 4307-4323. | |
| IL-4 | EP 0230107 | M23442 |
| IL-7 | EP 0314415 | J04156 |
| angiostatin | O′Reilly et al, Cell 79: 315-328 (1994) | X05199 |
| CEA | T. Barnett et al (1988) Genomics 3, 59-66. | M17303 |
| endostatin | O′Reilly et al., Cell 88: 277-285 (1997) | AF018081 |
| Tie2/Tek | Ziegler et al., Oncogene 8 (3), 663-670 (1993). | L06139 |
| receptor | | |
| 16 kD | Clapp et al., Endocrinology 133: 1292-1299 1993 | X54393 |
| fragment of | | |
| prolactin | | |
| angiopoietin-2 | Maisonpierre, P. C., et al., Science 277 (5322), 55-60 | AF004327 |
| | (1997) | |
| GM-CSF | G. G. Wong et al (1985). Human GM-CSF: molecular | E00951 |
| | cloning of the complementary DNA and purification of | |
| | the natural and recombinant proteins. Science 228, | |
| | 810-815. | |

This invention involves the use of one or more chimeric proteins, DNA constructs encoding them, and ligand molecules capable of triggering transcription of a target gene by binding to the ligand binding domain of the chimeric proteins. These are described in detail in the cited patent documents above. Briefly, the chimeric proteins contain at least one ligand-binding (or “receptor”) domain and an action domain capable of initiating transcription of the target gene within a cell. The chimeric proteins may further contain additional domains. These chimeric proteins and the constructs which encode them are recombinant in the sense that their various components are derived from different sources, and as such, are not found together in nature (i.e., are mutually heterologous). Also provided are recombinant target constructs containing a target gene under the transcriptional regulation of a transcriptional control element responsive to the presence of the ligand. The transcriptional control element is responsive in the sense that transcription of the target gene is activated by the presence of the ligand in cells containing these constructs. Said differently, exposure of the cells expressing the chimeric constructs and containing a target gene construct responsive to the ligand results in expression of the target gene. The constructs of this invention may contain one or more selectable markers such as a neomycin resistance gene (neor) and herpes simples virus-thymidine kinase (HSV-tk). When genetically engineered cells of this invention which contain and express the constructs, and contain the target gene construct, are exposed to the ligand, expression of the target gene is activated.

To render the mammal responsive to the ligand, certain of the mammal's cells must first be genetically engineered by introducing into them heterologous DNA constructs, typically in vivo. A variety of systems have been developed which permit the genetic engineering of cells to permit ligand-mediated regulatable expression of a target gene. See e.g. Clackson, “Controlling mammalian gene expression with small molecules”, Current Opinion in Chemical Biology 1:210-218 (1997). Materials and methods for implementing those systems are known in the art and may be adapted to the practice of the subject invention. Typically, at least two different heterologous DNA constructs are introduced into the cells, including (a) at least one target DNA construct which comprises a target gene, here a DNA sequence encoding a target protein, i.e., thrombospondin, angiostatin, endostatin, angiostatin-endostatin fusion proteins, angiopoietin, a soluble receptor for VEGF, a dominant negative form of VEGF, anti-VEGF antibodies, soluble Tie2/Tek receptor, the 16 kD fragment of prolactin, a tumor-specific antigen such as the carcinoembryonic antigen (CEA), a cytokine (such as IL-2, IL-4, IL-7, IL-12, GM-CSF, gamma interferon, etc) or beta-interferon, operably linked to a transcription control element permitting ligand-mediated expression of the target gene; and (b) one or more DNA constructs encoding and capable of directing the expression of chimeric proteins capable of binding to the ligand and activating expression of the target gene(s) in a ligand-dependent manner.

Preferred regulated expression systems are based on ligand-mediated dimerization of chimeric proteins. In such systems each of the chimeric proteins contains at least one ligand-binding (i.e., receptor) domain and at least one effector domain for activating gene transcription directly or indirectly. The phrase “ligand-binding domain” encompasses protein domains which are capable of binding to the ligand, as in the case of an FKBP domain and the ligand, FK506, discussed below, and further encompasses protein domains which are capable of binding to a complex of the ligand with another binding protein, as in the case of the FRB domain which binds to the rapamycin:FKBP complex. Examples of pairs of receptor domains and ligands which are known in the art and have been demonstrated to be effective in such regulated transcription systems, and which may be used in the practice of the subject invention, include FKBP/FK1012, FKBP/synthetic divalent FKBP ligands (see WO 96/0609 and WO 97/31898), FRB/rapamycin:FKBP (see e.g., WO 96/41865 and Rivera et al, “A humanized system for pharmacolgic control of gene expression”, Nature Medicine 2(9):1028-1032 (1997)), cyclophilin/cyclosporin (see e.g. WO 94/18317), DHFR/methotrexate (see e.g. Licitra et al, 1996, Proc. Natl. Acad. Sci. USA 93:12817-12821) and DNA gyrase/coumermycin (see e.g. Farrar et al, 1996, Nature 383:178-181).

In the case of direct activation of transcription, two chimeric proteins are typically used. Each, as mentioned above, contains at least one ligand-binding domain. One of the chimeras also contains at least one DNA-binding domain such as GAL4 or ZFHD1; the other contains at least one transcription activation domain such as VP16 or the p65 domain from NF-kappaB. The presence of a ligand to which the two chimeric proteins can bind, and through which the chimeric proteins can complex with one another to form protein dimers or multimers, activates transcription of a target gene linked to a transcription control element containing a DNA sequence which is recognized by, i.e., binds to, the DNA-binding domain. Typically the transcription control element also includes a minimal promoter sequence. DNA binding domains and transcription activation domains for use in treating human subjects preferably comprise human peptide sequence, as represented by ZFHD1 and p65. The transcription control element of a target gene construct to be directly activated by ligand-mediated dimerization will typically contain multiple copies of a recognition sequence for the DNA-binding domain and a minimal promoter. In embodiments involving a DNA binding domain (DBD), composite DBDs may be used with target gene constructs containing a corresponding binding site for the composite DBD, as described in the previously cited Gilman patent documents and in Pomerantz et al., PCT/US95/16982. In all such embodiments, multimerization activates transcription of a target gene under the transcriptional control of a transcriptional control element (e.g. enhancer and/or promoter elements and the like) which is responsive to the multimerization event.

In the case of systems for the indirect activation of transcription, at least one of the chimeric proteins also contains at least one ligand-binding domain and at least one effector domain. However, in these embodiments the effector domain comprises a cellular signaling domain such as the cytoplasmic domain of a growth factor receptor, which upon association with one or more like domains triggers transcription of a gene linked to a responsive promoter. Said differently, mutual association of such effector domains is considered to transmit an intracellular signal, which results in the activation of a responsive promoter. For example, clustering of the cytoplasmic portion of the zeta chain of the T Cell receptor triggers transcription of a gene linked to an IL-2 promoter. Numerous promoters responsive to the mutual association of various signaling domains are well known. See e.g. pages 23-26 of PCT/US94/01617 (WO 94/18317). The foregoing may be adapted to the subject invention to provide effector domains for the chimeric proteins and responsive promoters for target DNA constructs.

Alternatively, there are several ligand-mediated regulated transcription systems which are based on mechanisms other than ligand-mediated dimerization which may be adapted to the practice of the subject invention. In these systems, binding of ligand to a chimeric protein activates transcription of a target gene linked to a responsive transcription control sequence.

One such switch employs a deletion mutant of the human progesterone receptor which no longer binds progesterone or any known endogenous steroid but can be activated by the orally active progesterone antagonist RU486, described, e.g, in Wang et al. (1994) Proc. Natl. Acad. Sci. U.S.A. 91:8180. Thus, the chimeric protein consists of a ligand binding domain which binds RU486, a DNA binding domain such as GAL4 and a transcription activation domain such as VP16. Activation was demonstrated, e.g, in cells transplanted into mice using doses of RU486 (5-50 g/kg) considerably below the usual dose for inducing abortion in humans (10 mg/kg).

Another such system is referred to as the ecdysone inducible system. Early work demonstrated that fusing the Drosophila steroid ecdysone (Ec) receptor (EcR) Ec-binding domain to heterologous DNA binding and activation domains, such as E. coli lexA and herpesvirus VP16 permits ecdysone-dependent activation of target genes downstream of appropriate binding sites (Christopherson et al. (1992) Proc. Natl. Acad. Sci. U.S.A. 89:6314). An improved ecdysone regulation system has been developed, using the DNA binding domain of the EcR itself. In this system, the regulating transcription factor is provided as two proteins:

(1) a truncated, mutant EcR fused to herpes VP16 and (2) the mammalian homolog (RXR) of Ultraspiracle protein (USP), which heterodimerizes with the EcR (No et al. (1996) Proc. Natl. Acad. Sci. U.S.A. 93:3346). In this system, because the DNA binding domain was also recognized by a human receptor (the human famesoid X receptor), it was altered to a site recognized only by the mutant EcR. Thus, the invention provides an ecdysone inducible system, in which a truncated mutant EcR is fused to at least one subunit of a transcription activator of the invention. The transcription factor further comprises USP, thereby providing high level induction of transcription of a target gene having the EcR target sequence, dependent on the presence of ecdysone.

In another embodiment, the inducible system comprises the E. coli tet repressor (TetR), which binds to tet operator (tetO) sequences upstream of target genes. In the presence of tetracycline, or an analog, which bind to tetR, DNA binding is abolished and thus transactivation is abolished. This system, in which the TetR had previously been linked to transcription activation domains, e.g, from VP16, is generally referred to as an allosteric “off switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. Furthermore, depending on the concentration of the antibiotic in the culture medium (0-1 mu g/ml), target gene expression can be regulated over concentrations up to several orders of magnitude. Thus, the system not only allows differential control of the activity of an individual gene in eukaryotic cells but also is suitable for creation of “on/off” situations for such genes in a reversible way. This system provides low background and relatively high target gene expression in the absence of tetracycline or an analog. Thus, the invention described herein provides a method for obtaining even stronger transcription induction of a target gene, which is regulatable by the tetracycline system or other inducible DNA binding domain.

In another embodiment, a “reverse” Tet system is used, again based on a DNA binding domain that is a mutant of the E. coli TetR, but which binds to TetO in the presence of Tet.

For any of these systems, if used in the practice of the subject invention, it would be preferred to use a transcription activation domain of human origin, such as p65, in place of VP16. Additionally, it may be desirable to increase expression levels of the target gene by “bundling” of the activation domains, as described in Natesan et al., 09/126,009.

Depending on design preferences of the practitioner, a wide variety of ligands may be used. In general, ligands for use in this invention are preferably non-proteinaceous and preferably have a molecular weight below about 5 kD. Even more preferably, the ligand has a molecular weight of less than about 2 kDa, and even more preferably, less than 1500 Da. The ligand may bind to the chimeras in either order or simultaneously, preferably with a Kd value below about 10⁻⁶, more preferably below about 10⁻⁷, even more preferably below about 10⁻⁸, and in some embodiments below about 10⁻⁹ M. FK1012, cyclosporin-based divalent ligands, fujisporin and related types of semisynthetic ligands are disclosed in WO 94/18317 and PCT/US94/08008 (WO 95/02684). Ligands based on synthetic FKBP ligand monomers are disclosed in WO 96/06097 and WO 97/31898, and ligands based on rapamycin and derivatives are disclosed in WO 96/41865. Ligands for the ecdysone receptor, tet system and other proteins are disclosed in various cited references, including those cited and discussed above. All of the foregoing components may be used in the practice of this invention. Those documents also provide guidance in the design of constructs encoding such chimeras, expression vectors containing them, design and use of suitable target gene constructs and their use in engineering host cells.

FKBP, FRB, cyclophilin and other ligand binding domains comprising naturally occurring peptide sequence may be used in the design of chimeric proteins for use in practicing this invention. Alternatively, domains derived from naturally occurring sequences but containing one or more mutations in peptide sequence, generally at up to 10 amino acid positions, and preferably at 1-5 positions, more preferably at 1-3 positions and in some cases at a single amino acid residue, may be used in place of the naturally occurring counterpart sequence and can confer a number of important features. This is described at length in the previously cited patent documents, together with numerous examples of such mutations and corresponding ligands.

This invention further involves DNA vectors containing the various constructs described herein, whether for introduction into host cells in tissue culture, for introduction into embryos or for administration to whole organisms for the introduction of the constructs into cells in vivo. In either case the construct may be introduced episomally or for chromosomal integration. The vector may be a viral vector, including for example an adeno-, adeno associated- or retroviral vector. The constructs or vectors containing them may also contain selectable markers permitting selection of transfectants containing the construct.

This invention further encompasses the genetically engineered cells containing and/or expressing the constructs described herein, including prokaryotic and eucaryotic cells and in particular, yeast, worm, insect, mouse or other rodent, and other mammalian cells, including human cells, of various types and lineages, whether frozen or in active growth, whether in culture or in a whole organism containing them.

To recap, this invention provides materials and methods for regulatably expressing a target gene in engineered cells in response to the presence of a ligand which is added to the culture medium or administered to the whole organism, as the case may be. The method involves providing cells of this invention (or an organism containing such cells) which contain and are capable of expressing (a) one or more DNA constructs encoding one or more chimeric proteins capable, following exposure to ligand, of activating transcription of a target gene; and, (b) a target gene under the transcriptional regulation of an element responsive to binding of ligand to the ligand binding domain of the chimeric proteins. The method thus involves exposing the cells to a ligand capable of binding to the chimeric protein in an amount effective to result in detectable expression of the target gene. In cases in which the cells are growing in culture, exposure to the ligand is effected by adding the ligand to the culture medium. In cases in which the cells are present within a host organism, exposing them to the ligand is effected by administering the ligand to the host organism. For instance, in cases in which the host organism is an animal, in particular, a mammal the ligand is administered to the host animal by oral, bucal, sublingual, transdermal, subcutaneous, intramuscular, intravenous, intra-joint or inhalation administration in an appropriate vehicle therefor.

This invention further encompasses pharmaceutical or veterinary compositions for expressing a target gene in genetically engineered cells of this invention, including from animal tissue or from a subject containing such engineered cells. Such pharmaceutical or veterinary compositions comprise a ligand of this invention in admixture with a pharmaceutically or veterinarily acceptable carrier and optionally with one or more acceptable excipients. The ligand can be any ligand, so long as it is capable of binding to a chimeric protein(s) of this invention or triggering expression of the target gene in engineered cells of this invention. Likewise, this invention further encompasses a pharmaceutical or veterinary composition comprising an antagonist of this invention in admixture with a pharmaceutically acceptable carrier and optionally with one or more pharmaceutically or veterinarily acceptable excipients for preventing or reducing, in whole or part, the binding of ligand to the chimeric proteins in engineered cells of this invention, in cell culture or in a subject, and thus for preventing or reversing the activation of transcription of the target gene in the relevant cells. For example, one may use multimerization antagonists which reduce or prevent multimerization of the chimeric proteins. Thus, the use of the reagents and of the antagonist reagents to prepare pharmaceutical or veterinary compositions is encompassed by this invention.

This invention also offers a method for providing a host organism, preferably an animal, and in many cases a mammal, susceptible to regulatable expression of a target gene in response to a ligand of this invention. The method involves introducing into the organism cells which have been engineered ex vivo in accordance with this invention, i.e. containing a DNA construct encoding a chimeric protein hereof, and so forth. Alternatively, one can introduce the DNA constructs of this invention into a host organism, e.g. mammal or embryo thereof, under conditions permitting transduction of one or more cells of the host mammal in vivo.

Design and Assembly of the Constructs

Constructs may be designed in accordance with the principles, illustrative examples and materials and methods disclosed in the patent documents and scientific literature cited herein, with modifications and further exemplification as described herein. Components of the constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. In the case of DNA constructs encoding fusion proteins, DNA sequences encoding individual domains and sub domains are joined such that they constitute a single open reading frame encoding a fusion protein capable of being translated in cells or cell lysates into a single polypeptide harboring all component domains. The DNA construct encoding the fusion protein may then be placed into a vector that directs the expression of the protein in the appropriate cell type(s). For biochemical analysis of the encoded chimera, it may be desirable to construct plasmids that direct the expression of the protein in bacteria or in reticulocyte-lysate systems. For use in the production of proteins in mammalian cells, the protein-encoding sequence is introduced into an expression vector that directs expression in these cells. Expression vectors suitable for such uses are well known in the art. Various sorts of such vectors are commercially available.

Antisense Messages and Ribozymes for Blocking HIV Gene Expression

Any gene sequence of an infectious agent such as HIV may be targeted to prevent its expression using ligand-regulated expression of antisense message or ribozyme. An antisense message or a ribozyme contains sufficient sequence complementary to the target gene such that it specifically recognizes the target message and blocks its expression. For a recent review containing useful background information and guidance, see Altman, 1993, RNA enzyme-directed gene therapy, Proc. Natl. Acad. Sci. USA 90, 10898-10900 and papers cited therein, including Yu et al., 1993, A hairpin ribozyme inhibits expression of diverse strains of human immunodeficiency virus type 1.,Proc. Natl. Acad. Sci. USA 90, 6340-6344. See also Efrat et al., Ribozyme-mediated attenuation of pancreatic β-cell glucokinase expression in transgenic mice results in impaired glucose-induced insulin secretion.,Proc. Natl. Acad. Sci. USA 91, 2051-2055.

Interference by a Dominant Negative Gene Product

Protein-protein interactions that are critical for a cellular process can be selectively blocked by expression of a non-functional variant of one of the protein partners. For example, raf-1 is a serine/threonine protein kinase that functions in growth factor-stimulated proliferation pathways (Schaap et al. J. Biol. Chem. 268: 20232 1993). It is composed of two domains, an N-terminal regulatory domain and C-terminal kinase domain. Constitutive overexpression of the N-terminal domain of p74raf-1 in cultured cells blocked mitogenesis induced by growth factors. This domain also interfered with an oncogenic variant of p21ras. Such a system could be useful for models of cancer or the role of growth factors on cellular proliferation. Similarly, the transformed phenotype of certain cell types can be reverted or suppressed by the expression of dominant-negative growth factors such as PDGF-O (Vassbotn et al., Mol Cell Biol. 13:4066-4076, 1993).

Other examples of dominant negative gene products include certain variants of steroid receptors, growth factor receptors having an inactive protein kinase or lacking the protein kinase domain altogether, cell surface receptors having a non-functional extracellular ligand binding domain or intracellular cytoplasmic domain, transcription factor variants that lack a DNA binding domain and/or a transactivation domain.

Dominant negative proteins typically disrupt the normal function of a target protein by sequestering it away from its normal partner. Constructs encoding dominant negative variants of VEGF for use in cancer applications can be constructed by random mutagenesis, by selective deletion of gene segments, or by a rational protein engineering.

One important requirement is that the dominant negative protein be overexpressed relative to its normal counterpart. The increased expression afforded by the ligand-regulated transcriptional activation of our invention makes this a particularly useful application of the technology.

Applications of Target Genes Encoding a Dominant Negative Variant, Ribozyme or Antisense Message Directed Against an HIV Target Gene

Intracellular immunization is the process of transfering a gene into a cell that protects that cell from a harmful agent, which can be either physical (i.e., irradiation), chemical (i.e., chemotherapeutic drugs), or biological (i.e., infectious agents such as viruses). Intracellular immunization is a particular relevant for treatment or prevention of AIDS, which is caused by the spread of HIV virus in cells of the blood. To treat or prevent HIV infection by intracellular immunization, a patient's blood cells may be transduced with a suitably engineered gene. This gene may be introduced into peripheral blood cells, preferably into progenitor cells of the hematopoietic system, more preferably into totipotent hematopoietic stem cells, through the use of physical DNA transfer methods or viral vectors, such that all offspring of the cells carry the engineered gene and express the encoded gene product. The gene product is a protein or RNA that either blocks establishment of an HIV infection or prevents production of infectious virus from previously-infected cells. Thus, intracellular immunization is expected to reduce virus load, halt the death of CD4 lymphocytes, and prevent the degeneration of immune system function that is the basis for morbidity and mortality in AIDS.

Examples of candidate agents for inducing intracellular immunization against a virus such as HIV include antisense RNA, ribozymes that cleave viral RNA, dominant-negative viral proteins (e.g., dominant-negative Tat or Rev proteins for HIV), intracellular antibodies directed against viral proteins, and capsid-nuclease fusion proteins. A common feature of these agents is that they act at least in part stoichiometrically, either by competition, hybridization, or incorporation into multi-component complexes. Therefore, high intracellular concentrations of these agents is essential to their efficacy.

Regulated gene therapy permits the controlled high-level expression of intracellular proteins of this type and therefore will augment the efficacy of intracellular immunization agents in actual practice: An additional advantage of regulated gene therapy for this application is enhanced safety. The potential cellular toxicities of these agents are not yet known; regulated gene therapy permits production to be kept below levels associated with toxicity. Finally, regulated gene therapy permits treatment with the intracellular immunization agent to be terminated when the patient is free of danger and restored at a later time, if needed.

The use of regulated gene therapy for producing intracellular immunization may be essential for the successful implementation of stem cell-based gene therapy, because, once administered to the patient, engineered stem cells and their progeny cannot be recovered. The only mechanism for reversing therapy is termination of production of the therapeutic agent. Therefore, regulated gene therapy greatly improves the prospects for intracellular immunization.

Soluble VEGF Receptor

A construct encoding a soluble VEGF receptor may be prepared using conventional methods such as were used in other soluble receptor examples. For instance, the extracellular ligand binding domain of the VEGF receptor may be expressed and purified using the cloned receptor cDNA. Identification of the receptor extracellular domain can be done by performing a Kyte-Doolittle analysis on the coding sequence. In the case of cytokine and growth factor receptors, the extracellular domain is N-terminal of the transmembrane spanning (TM) domain. The TM domain marks the end of the ligand binding domain and in the Kyte-Doolittle profile is demarked by a high hydrophobicity index over a span of between 20-30 amino acids. For an example of the Kyte-Doolittle analysis of the EPO-receptor see U.S. Pat. No. 5,278,065. To produce the ligand binding domain of a receptor, i.e., a soluble receptor, the cDNA encoding the extracellular domain is cloned into an appropriate expression vector such as pET11a (Invitrogen) for E. coli, pVL1393 (Invitrogen) for insect cells, or pcDNA (Invitrogen) for mammalian cells. A stop codon is introduced at/before the first amino acid of the TM domain.

When this so-called soluble receptor is expressed in yeast, insect cells or mammalian cells, the protein is secreted into the cell culture medium (see Kikuchi et al J. Immunol. Methods 167:289 1994). Alternatively, when the ligand binding domain is expressed in E. coli, the soluble receptor collects in the periplasmic space (see Cunningham et al Science 254: 821 1991). To facilitate purification and binding assays the extracellular domain may be expressed fused to an epitope tag such as the epitope for the anti-myc antibody 9E10 or the “Flag” epitope (IBI) (see Kolodziej and Young, Methods Enzymol 194: 508 (1991)). Alternatively, the ligand binding domain may be expressed fused to the heavy chain of an immunoglobulin as described in (Ashkenazi et al PNAS 88:10535 1991). The ligand binding domain can be expressed in E. coli, yeast, insect cells, mammalian cells or produced using an in vitro transcription/translation system (Promega). Expression in mammalian cells can be accomplished using transient expression or by stable selection of clones using a selectable drug such as G418. For details of expression systems see Goeddel (ed.) Methods Enzymol vol 185 1990). Purification of the expressed protein can be accomplished by standard chromatographic methods, by ligand affinity chromatography or by means of the fusion partner such as an antibody epitope or immunoglobulin heavy chain.

Soluble receptors for VEGF have been cloned by the methods described above. For example, sFLT was cloned from a human unbilical vein endothelial cell library by plaque screening and PCR as described in Kendall et al., PNAS USA 90:10705-10709, November 1993. Other groups have cloned soluble receptors by alternative methods. See, for example, Lin et al., Cell Growth and Differentiation, 9:49-58 January 1998, in which the extracellular domain of murine flk-1 was amplified by PCR. Millauer et al. (Nature 367:576-579 Feb. 10, 1994) report the dominant negative inhibition of Flk-1 activity by formation of signalling-defective heterodimers comprising the Flk-1 transmembrane domain.

Tissue-Specific or Cell-Type Specific Expression

It will be preferred in certain embodiments, that the chimeric proteins be expressed in a cell-specific or tissue-specific manner. Such specificity of expression may be achieved by operably linking one or more of the DNA sequences encoding the chimeric protein(s) to a cell-type specific transcriptional regulatory sequence (e.g. promoter/enhancer). Numerous cell-type specific transcriptional regulatory sequences are known. Others may be obtained from genes which are expressed in a cell specific manner. See e.g. PCT/US95/10591, especially pp. 36-37.

For example, constructs for expressing the chimeric proteins may contain regulatory sequences derived from known genes for specific expression in selected tissues. Representative examples are tabulated below:

| Tissue | Gene | Reference |
|---|---|---|
| lens | g2 crystallin | Breitman, M. L., Clapoff, S., |
| | | Rossant, J., Tsui, L. C., Golde, L. M., |
| | | Maxwell, I. H., Bernstin, |
| | | A. (1987) Genetic Ablation: |
| | | targeted expression of a toxin |
| | | gene causes microphthalmia in |
| | | transgenic mice. Science 238: |
| | | 1563-1565 |
| | aA crystallin | Landel, C. P., Zhao, J., Bok, D., |
| | | Evans, G. A. (1988) Lens- |
| | | specific expression of a |
| | | recombinant ricin induces |
| | | developmental defects in the |
| | | eyes of transgenic mice. Genes |
| | | Dev. 2: 1168-1178 |
| | | Kaur, S., key, B., Stock, J., |
| | | McNeish, J. D., Akeson, R., |
| | | Potter, S. S. (1989) Targeted |
| | | ablation of alpha-crystallin- |
| | | synthesizing cells produces |
| | | lens-deficient eyes in |
| | | transgenic mice. Development |
| | | 105: 613-619 |
| pituitary - somatrophic cells | Growth hormone | Behringer, R. R., Mathews, L. S., |
| | | Palmiter, R. D., Brinster, R. L. |
| | | (1988) Dwarf mice produced |
| | | by genetic ablation of growth |
| | | hormone expressing cells. |
| | | Genes Dev. 2: 453-461 |
| pancreas | Insulin- | Ornitz, D. M., Palmiter, R. D., |
| | Elastase - acinar cell specific | Hammer, R. E., Brinster, R. L., |
| | | Swift, G. H., MacDonald, R. J. |
| | | (1985) Specific expression of |
| | | an elastase human growth |
| | | fusion in pancreatic acinar |
| | | cells of transgeneic mice. |
| | | Nature 131: 600 603 |
| | | Palmiter, R. D., Behringer, R. R., |
| | | Quaife, C. J., Maxwell, F., |
| | | Maxwell, I. H., Brinster, R. L. |
| | | (1987) Cell lineage ablation in |
| | | transgeneic mice by cell- |
| | | specific expression of a toxin |
| | | gene. Cell 50: 435-443 |
| T cells | lck promoter | Chaffin, K. E., Beals, C. R., |
| | | Wilkie, T. M., Forbush, K. A., |
| | | Simon, M. I., Perlmutter, R. M. |
| | | (1990) EMBO Journal 9: 3821-3829 |
| B cells | Immunoglobulin kappa light | Borelli, E., Heyman, R., Hsi, M., |
| | chain | Evans, R. M. (1988) |
| | | Targeting of an inducible toxic |
| | | phenotype in animal cells. |
| | | Proc. Natl. Acad. Sci. USA |
| | | 85: 7572-7576 |
| | | Heyman, R. A., Borrelli, E., |
| | | Lesley, J., Anderson, D., |
| | | Richmond, D. D., Baird, S. M., |
| | | Hyman, R., Evans, R. M. |
| | | (1989) Thymidine kinase |
| | | obliteration: creation of |
| | | transgenic mice with |
| | | controlled immunodeficiencies. |
| | | Proc. Natl. Acad. Sci. USA |
| | | 86: 2698-2702 |
| Schwann cells | P0 promoter | Messing, A., Behringer, R. R., |
| | | Hammang, J. P. Palmiter, RD, |
| | | Brinster, RL, Lemke, G., P0 |
| | | promoter directs espression of |
| | | reporter and toxin genes to |
| | | Schwann cells of transgenic |
| | | mice. Neuron 8: 507-520 1992 |
| | Myelin basic protein | Miskimins, R. Knapp, L., |
| | | Dewey, MJ, Zhang, X. Cell and |
| | | tissue-specific expression of a |
| | | heterologous gene under |
| | | control of the myelin basic |
| | | protein gene promoter in |
| | | trangenic mice. Brain Res Dev |
| | | Brain Res 1992 Vol 65: 217-21 |
| spermatids | protamine | Breitman, M. L., Rombola, H., |
| | | Maxwell, I. H., Klintworth, G. K., |
| | | Bernstein, A. (1990) |
| | | Genetic ablation in transgenic |
| | | mice with attenuated |
| | | diphtheria toxin A gene. Mol. |
| | | Cell. Biol. 10: 474-479 |
| adipocyte | P2 | Ross, S. R, Braves, RA, |
| | | Spiegelman, BM Targeted |
| | | expression of a toxin gene to |
| | | adipose tissue: transgenic mice |
| | | resistant to obesity Genes and |
| | | Dev 7: 1318-24 1993 |
| muscle | myosin light chain | Lee, KJ, Ross, RS, Rockman, |
| | | HA, Harris, AN, O'Brien, TX, |
| | | van-Bilsen, M., Shubeita, HE, |
| | | Kandolf, R., Brem, G., Prices |
| | | et al J. BIol. Chem. 1992 Aug 5, |
| | | 267: 15875-85 |
| | Alpha actin | Muscat, GE., Perry, S., |
| | | Prentice, H. Kedes, L. The |
| | | human skeletal alpha-actin |
| | | gene is regulated by a muscle- |
| | | specific enhancer that binds |
| | | three nuclear factors. Gene |
| | | Expression 2, 111-26, 1992 |
| liver | tyrosine aminotransferase, | |
| | albumin, | |
| | apolipoproteins | |
| neurons | neurofilament proteins | Reeben, M. Halmekyto, M. |
| | | Alhonen, L. Sinervirta, R. |
| | | Saarma, M. Janne, J. Tissue |
| | | specific expression of rat light |
| | | neurofilament promoter-driven |
| | | reporter gene in transgenic |
| | | mice. BBRC 1993: 192: 465-70 |
| lung | Lung surfacant gene | Ornitz, D. M., Palmiter, R. D., |
| | | Hammer, R. E., Brinster, R. L., |
| | | Swift, G. H., MacDonald, R. J. |
| | | (1985) Specific expression of |
| | | an elastase human growth |
| | | fusion in pancreatic acinar |
| | | cells of transgeneic mice. |
| | | Nature 131: 600 603 |

Introduction of Constructs into Cells

Constructs encoding the chimeric protein molecules and constructs directing the expression of target genes, all as described herein, can be introduced into cells as one or more DNA molecules or constructs, in many cases in association with one or more markers to allow for selection of host cells which contain the construct(s). The constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. The construct(s) once completed and demonstrated to have the appropriate sequences may then be introduced into a host cell by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells will in some cases be grown and expanded in culture before introduction of the construct(s), followed by the appropriate treatment for introduction of the construct(s) and integration of the construct(s). The cells will then be expanded and screened by virtue of a marker present in the construct. Various markers which may be used successfully include hprt, neomycin resistance, thymidine kinase, hygromycin resistance, etc.

In some instances, one may have a target site for homologous recombination, where it is desired that a construct be integrated at a particular locus. For example, one can delete and/or replace an endogenous gene (at the same locus or elsewhere) with a recombinant target construct of this invention. For homologous recombination, one may generally use either Ω or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding all of the genes, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in procaryotes or eucaryotes, etc. which may be used to prepare stocks of construct DNAs and for carrying out transfections are well known in the art, and many are commercially available.

Introduction of Constructs into host Organisms

Cells which have been modified ex vivo with the DNA constructs may be grown in culture under selective conditions and cells which are selected as having the desired construct(s) may then be expanded and further analyzed, using, for example, the polymerase chain reaction for determining the presence of the construct in the host cells. Once modified host cells have been identified, they may then be used as planned, e.g. grown in culture or introduced into a host organism.

Depending upon the nature of the cells, the cells may be introduced into a host organism, e.g. a mammal, in a wide variety of ways. Hematopoietic cells may be administered by injection into the vascular system, there being usually at least about 10⁴ cells and generally not more than about 10¹⁰, more usually not more than about 10⁸ cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Alternatively, with skin cells which may be used as a graft, the number of cells would depend upon the size of the layer to be applied to the burn or other lesion. Generally, for myoblasts or fibroblasts, the number of cells will be at least about 10⁴ and not more than about 10⁸ and may be applied as a dispersion, generally being injected at or near the site of interest. The cells will usually be in a physiologically-acceptable medium.

Cells engineered in accordance with this invention may also be encapsulated, e.g. using conventional materials and methods. See e.g. Uludag and Sefton, 1993, J. Biomed. Mater. Res. 27(10):1213-24; Chang et al, 1993, Hum Gene Ther 4(4):433-40; Reddy et al, 1993, J Infect Dis 168(4):1082-3; Tai and Sun, 1993, FASEB J 7(11):1061-9; Emerich et al, 1993, Exp Neurol 122(1):37-47; Emerich et al, 1994, Exp Neurol 130:141-150; Sagen et al, 1993, J Neurosci 13(6):2415-23; Aebischer et al, 1994, Exp Neurol 126(2):151-8; Savelkoul et al, 1994, J Immunol Methods 170(2):185-96; Winn et al, 1994, PNAS USA 91(6):2324-8; Emerich et al, 1994, Prog Neuropsychopharmacol Biol Psychiatry 18(5):935-46; Emerich et al, J Comparative Neurology 349:148-164 (1994); Joseph, 1994, Cell Transplanation 3(5):355-364 and Kordower et al, 1994, PNAS USA 91(23):10898-902. The cells may then be introduced in encapsulated form into an animal host, preferably a mammal and more preferably a human subject in need thereof. Preferably the encapsulating material is semipermeable, permitting release into the host of secreted proteins produced by the encapsulated cells. In many embodiments the semipermable encapsulation renders the encapsulated cells immunologically isolated from the host organism in which the encapsulated cells are introduced. In those embodiments the cells to be encapsulated may express one or more chimeric proteins containing component domains derived from viral proteins or proteins from other species (and no longer preferably contain a composite DNA binding domain as described in detail in the Gilman references, supra). For example in those cases the chimeras may well contain elements derived from GAL4 and VP16. In such cases, the cells may be engineered as disclosed in International Patent Applications PCT/US94/01617 or PCT/US94/08008 or in WO96/06111.

Instead of ex vivo modification of the cells, in many situations one may wish to modify cells in vivo. For this purpose, various techniques have been developed for modification of target tissue and cells in vivo. A number of virus vectors have been developed, such as adenovirus, adeno-associated virus and retroviruses, which allow for transfection and random integration of the virus into the host, as described below.

Adenoviral Vectors:

A viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. In contrast to retrovirus, the infection of adenoviral DNA into host cells does not result in chromosomal integration because adenoviral DNA replicates in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in the human.

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them preferred mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Adenovirus vectors have also been used in vaccine development (Grunha us and Horwitz (1992) Siminar in Virology 3:237; Graham and Prevec (1992) Biotechnology 20:363). Experiments in administering recombinant adenovirus to different tissues include trachea instillation (Rosenfeld et al. (1991); Rosenfeld et al. (1992) Cell 8:143), muscle injection (Ragot et al. (1993) Nature 361:647), peripheral intravenous injection Herz and Gerard (1993) Proc. Natl. Acad. Sci. U.S.A. 90:2812), and stereotactic inoculation into the brain (Le Gal La Salle et al. (1993) Science 254:988).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the method of the present invention. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the nucleic acid of interest in a region within the adenovirus sequences is not critical to the present invention. For example, the nucleic acid of interest may also be inserted in lieu of the deleted E3 region in E3 replacement vectors as described previously by Karlsson et. al. (1986) or in the E4 region where a helper cell line or helper virus complements the E4 defect.

A preferred helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J. General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Various adenovirus vectors have been shown to be of use in the transfer of genes to mammals, including humans. Replication-deficient adenovirus vectors have been used to express marker proteins and CFTR in the pulmonary epithelium. Because of their ability to efficiently infect dividing cells, their tropism for the lung, and the relative ease of generation of high titer stocks, adenoviral vectors have been the subject of much research in the last few years, and various vectors have been used to deliver genes to the lungs of human subjects (Zabner et al., Cell 75:207-216, 1993; Crystal, et al., Nat Genet. 8:42-51, 1994; Boucher, et al., Hum Gene Ther 5:615-639, 1994). The first generation E1a deleted adenovirus vectors have been improved upon with a second generation that includes a temperature-sensitive E2a viral protein, designed to express less viral protein and thereby make the virally infected cell less of a target for the immune system (Goldman et al., Human Gene Therapy 6:839 851,1995). More recently, a viral vector deleted of all viral open reading frames has been reported (Fisher et al., Virology 217:11-22, 1996). Moreover, it has been shown that expression of viral IL-b10 inhibits the immune response to adenoviral antigen (Qin et al., Human Gene Therapy 8:1365-1374, 1997).

Adenoviruses can also be cell type specific, i.e., infect only restricted types of cells and/or express a transgene only in restricted types of cells. For example, the viruses comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1B.

DNA sequences of a number of adenovirus types are available from Genbank. For example, human adenovirus type 5 has GenBank Accession No.M73260. The adenovirus DNA sequences may be obtained from any of the 42 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by the same methods (restriction digest, linker ligation or filling in of ends, and ligation) used to insert the CFTR or other genes into the vectors.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.;PCT/FR94/00851 (WO 95/02697) by Perrocaudet et al., PCT/US95/14793 (WO96/14061) by Wang et al.

AAV Vectors:

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector.

AAV is also one of the few viruses that may integrate its DNA into non-dividing cells, e.g., pulmonary epithelial cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611 619; and Flotte et al., (1993) J. Biol. Chem. 268:3781-3790).

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The following proteins have been expressed using various AAV-based vectors: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A transgene incorporating the various DNA constructs of this invention can similarly be included in an AAV-based vector. As an alternative to inclusion of a constitutive promoter such as CMV to drive expression of the recombinant DNA encoding the chimeric protein(s), e.g. chimeric proteins comprising an activation domain or DNA-binding domain, an AAV promoter can be used (ITR itself or AAV p5 (Flotte, et al. J. Biol. Chem. 268:3781-3790, 1993)).

Such a vector can be packaged into AAV virions by reported methods. For example, a human cell line such as 293 can be co transfected with the AAV-based expression vector and another plasmid containing open reading frames encoding AAV rep and cap (which are obligatory for replication and packaging of the recombinant viral construct) under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B. J., Current Opinion in Biotechnology 3:533-539, 1992; Kotin, R. M, Human Gene Therapy 5:793-801, 1994)). Typically, three days after transfection, recombinant AAV is harvested from the cells along with adenoviurs and the contaminating adenovirus is then inactivated by heat treatment.

Methods to improve the titer of AAV can also be used to express the transgene in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997. In yet another method for producing large amounts of recombinant AAV, a chimeric plasmid is used which incorporate the Epstein Barr Nuclear Antigen (EBNA) gene, the latent origin of replication of Epstein Barr virus (oriP) and an AAV genome. These plasmids are maintained as a multicopy extra-chromosomal elements in cells, such as in 293 cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug. 19, 1997). Additionally, one may increase the efficiency of AAV transduction by treating the cells with an agent that facilitates the conversion of the single stranded form to the double stranded form, as described in Wilson et al., WO96/39530.

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO97/08298.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

For additional detailed guidance on AAV technology which may be useful in the practice of the subject invention, including methods and materials for the incorporation of a transgene, the propagation and purification of the recombinant AAV vector containing the transgene, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146(25 Jul. 1995); Chatterjee et al, U.S. Pat. No. 5,454,935 (12 Dec. 1995), Carter et. al WO 93/24641 (published 9 Dec. 1993), and Natsoulis, U.S. Pat. No. 5,622,856 (Apr. 22, 1997). Further information regarding AAVs and the adenovirus or herpes helper functions required can be found in the following articles: Berns and Bohensky (1987), “Adeno-Associated Viruses: An Update”, Advances in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does not require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

Hybrid Adenovirus-AAV Vectors:

Hybrid Adenovirus-AAV vectors represented by an adenovirus capsid containing a nucleic acid comprising a portion of an adenovirus, and 5 and 3′ ITR sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in the this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the AdS genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100.

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected transgene. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

Retroviruses:

The retroviruses are a group of single-stranded RNA viruses characterized by an ability to convert their RNA to double-stranded DNA in infected cells by a process of reverse-transcription (Coffin (1990) Retroviridae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsial proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Exprssion of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types. However, integration and stable expression require the division of host cells (Paskind et al. (1975) Virology 67:242).

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, e.g., a transcriptional activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460 6464; Wilson et al., (1988) PNAS USA 85:3014-3018; Armentano et al., (1990) PNAS USA 87:6141-6145; Huber et al., (1991) PNAS USA 88:8039 8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Other Viral Systems:

Other viral vector systems that may have application in gene therapy have been derived from herpes virus, e.g., Herpes Simplex Virus (U.S. Pat. No. 5,631,236 by Woo et al., issued May 20, 1997), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. In particular, herpes virus vectors may provide a unique strategy for persistence of the recombinant gene in cells of the central nervous system and ocular tissue (Pepose et al., (1994) Invest Ophthalmol Vis Sci 35:2662-2666). They offer several attractive features for various mammalian cells (Friedmann (1989) Science, 244:1275-1281; Ridgeway, 1988, supra; Baichwal and Sugden, 1986, supra; Coupar et al., 1988; Horwich et al.(1990) J.Virol., 64:642-650).

With the recent recognition of defective hepatitis B viruses, new insight was gained into the structure-function relationship of different viral sequences. In vitro studies showed that the virus could retain the ability for helper-dependent packaging and reverse transcription despite the deletion of up to —80% of its genome (Horwich et al., 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

Administration of Viral Vectors:

Generally the DNA or viral particles are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles. Delivery of the transgene as naked DNA; as lipid-, liposome-, or otherwise formulated DNA; or as a recombinant viral vector is then preferably carried out via in vivo, lung-directed, gene therapy. This can be accomplished by various means, including nebulization/inhalation or by instillation via bronchoscopy. Recently, recombinant adenovirus encoding CFTR was administered via aerosol to human subjects in a phase I clinical trial. Vector DNA and CFTR expression were clearly detected in the nose and airway of these patients with no acute toxic effects (Bellonet al., Human Gene Therapy, 8(1):15-25, 1997).

Preferably, the DNA or recombinant virus is administered insufficient amounts to transfect cells within the recipient's airways, including without limitation various airway epithelial cells, leukocytes residing within the airways and accessible airway smooth muscle cells, and provide sufficient levels of transgene expression to provide for observable ligand-responsive transcription of a target gene, preferably at a level providing therapeutic benefit without undue adverse effects.

Optimal dosages of DNA or virus depends on a variety of factors, as discussed previously, and may thus vary somewhat from patient to patient. Again, therapeutically effective doses of viruses are considered to be in the range of about 20 to about 50 ml of saline solution containing concentrations of from about 1×10⁷ to about 1×10¹⁰ pfu of virus/ml, e.g. from 1×10⁸ to 1×10⁹ pfu of virus/ml.

In a preferred embodiment, the ratio of viral particle containing a target gene versus viral particles containing nucleic acids encoding the chimeric proteins of the invention is about 1:1. However, other ratios can also be used. For example, in certain instances it may be desirable to administer twice as many particles having the target gene as those encoding the chimeric proteins. Other ratios include 1:3, 1:4, 1:10, 2:1, 3:1, 4:1, 5:1, 10:1. The optimal ratio can be determined by performing in vitro assays using the different ratios of viral particles to determine which ratio results in highest expression and lowest background expression of the target gene. Similarly, in situations in which the chimeric proteins are encoded by two different nucleic acids each encapsidated separately, one can vary the ratio between the three viral particles, according to the result desired.

In accordance with in vivo genetic modification, the manner of the modification will depend on the nature of the tissue, the efficiency of cellular modification required, the number of opportunities to modify the particular cells, the accessibility of the tissue to the DNA composition to be introduced, and the like. By employing an attenuated or modified retrovirus carrying a target transcriptional initiation region, if desired, one can activate the virus using one of the subject transcription factor constructs, so that the virus may be produced and transfect adjacent cells.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The ligand may be administered to the patient as desired to activate transcription of the target gene. Depending upon the binding affinity of the ligand, the response desired, the manner of administration, the half-life, the number of cells present, various protocols may be employed. The ligand may be administered parenterally or orally. The number of administrations will depend upon the factors described above. The ligand may be taken orally as a pill, powder, or dispersion; bucally; sublingually; injected intravascularly, intraperitoneally, subcutaneously; by inhalation, or the like. The ligand (and monomeric antagonist compound) may be formulated using conventional methods and materials well known in the art for the various routes of administration. The precise dose and particular method of administration will depend upon the above factors and be determined by the attending physician or human or animal healthcare provider. For the most part, the manner of administration will be determined empirically.

In the event that transcriptional activation by the ligand is to be reversed or terminated, a compound which can compete with the ligand may be administered. Thus, in the case of an adverse reaction or the desire to terminate the therapeutic effect, an antagonist can be administered in any convenient way, particularly intravascularly, if a rapid reversal is desired. Alternatively, one may provide for the presence of an inactivation domain (or transcriptional silencer) with a DNA binding domain. In another approach, cells may be eliminated through apoptosis via signalling through Fas or TNF receptor as described elsewhere. See e.g.,PCT/US94/01617 and PCT/US94/08008.

The particular dosage of the ligand for any application may be determined in accordance with the procedures used for therapeutic dosage monitoring, where maintenance of a particular level of expression is desired over an extended period of times, for example, greater than about two weeks, or where there is repetitive therapy, with individual or repeated doses of ligand over short periods of time, with extended intervals, for example, two weeks or more. A dose of the ligand within a predetermined range would be given and monitored for response, so as to obtain a time-expression level relationship, as well as observing therapeutic response. Depending on the levels observed during the time period and the therapeutic response, one could provide a larger or smaller dose the next time, following the response. This process would be iteratively repeated until one obtained a dosage within the therapeutic range. Where the ligand is chronically administered, once the maintenance dosage of the ligand is determined, one could then do assays at extended intervals to be assured that the cellular system is providing the appropriate response and level of the expression product. Thus, in each case, the patient would be monitored for the proper dosage for that individual.

Biological Research

This invention is also applicable to a wide range of biological experiments in which precise control over a target gene is desired. These include: (1) expression of a protein or RNA of interest for biochemical purification; (2) regulated expression of a protein or RNA of interest in tissue culture cells for the purposes of evaluating its biological function; (3) regulated expression of a protein or RNA of interest in transgenic animals for the purposes of evaluating its biological function; (4) regulating the expression of another regulatory protein that acts on an endogenous gene for the purposes of evaluating the biological function of that gene.

The full contents of all references cited in this document, including references from the scientific literature, issued patents and published patent applications, are hereby expressly incorporated by reference.

The following examples contain important additional information, exemplification and guidance which can be adapted to the practice of this invention in its various embodiments and the equivalents thereof. The examples are offered by way of illustration and should not be construed as limiting in any way. As noted throughout this document, the invention is broadly applicable and permits a wide range of design choices by the practitioner.

The practice of this invention will employ, unless otherwise indicated, conventional techniques of cell biology, cell culture, molecular biology, transgenic biology, microbiology, recombinant DNA, immunology, virology, pharmacology, chemistry, and pharmaceutical formulation and administration which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonudeotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

The following experimental details are provided as further guidance to the practitioner. Information and approaches for subcloning, assembly of constructs and vectors and various components may be useful in the design and construction of transgenes for use in the practice of this invention.

### Example 1

### Construction of Transcription Factor and Target Gene Vectors

A. Direct Activation of Transcription

Transcription Factor Plasmid:

pCEN-F3p65/Z1F3/neo

Transcription factor fusion proteins and the neo gene are expressed from the mammalian expression vector pCEN, a derivative of pCGNN (7, 8). Inserts cloned into pCEN as XbaI-BamHI fragments are transcribed under control of the human CMV enhancer/promoter (C) and are expressed with an amino-terminal epitope tag (E, a 16 amino acid portion of the influenza hemagglutin [HA] gene) and nuclear localization sequence (N) from the SV40 large T antigen. pCEN-F3p65/Z1F3/neo produces a tricistronic transcript encoding the activation domain fusion 3×FKBP-p65 (F3p65), the DNA binding domain fusion ZFHD1-3×FKBP (Z1F3), and the neo gene, each separated by an internal ribosome entry sequence (IRES) from the encephalomyocarditis virus (see below). For human gene therapy applications, epitope tags are preferably omitted.

Target Plasmids:

LH-Z12-1-PL

This plasmid/retroviral vector contains long terminal repeats (LTRs) from the Moloney murine leukemia virus, one of which drives expression of the hygromycin resistance gene (see (3, 9)). Downstream of the hygromycin gene are 12 ZFHD1 binding sites, a minimal human interleukin-2 (IL2) gene promoter and a polylinker. Insertion of the gene of interest into the polylinker puts its expression under control of the dimerizer-regulated transcription factors. Despite the presence of enhancers within the LTRs, in cell lines tested, this vector has low basal expression (3). Note that this vector can be used directly as a plasmid for transient transfections and for generating stable cell lines or it can be used to make retrovirus (see below). It is sufficient—and preferable when the vector is used to generate retrovirus—to insert only the coding sequence of the gene to be regulated, without the poly(A) signal or introns.

Alternatively, the ZFHD1-IL2 control region can be removed from this vector (using 5′ MluI or NheI sites and 3′ HindIII, PstI, EcoRI, SpeI, BglII or ClaI sites) and inserted upstream of the gene of interest. This may be preferred when it is important to maintain the genomic structure of the gene.

LH-Z12-1-S

This control vector contains the secreted alkaline phosphatase gene (a HindIII-ClaI fragment from pSEAP promoter vector, Clontech) inserted into LH-Z12-1-PL.

General Information

Procedure for Making Stable Cell Lines

I. Stably Integrate the Regulated Transcription Factors

A. Transfect Cells with pCEN-F3p65/ZlF3/neo

Linearization with SfiI enhances the efficiency of integration.

B. Select G418-Resistant Clones

>90% of G418-resistant clones should express the transcription factors.

C. Screen by Transient Transfection with LH-Z12-1-S (or Another Easily Assayed Reporter Plasmid) for Clones with Low Background and High Dimerizer-Dependent Induction

The absolute level of inducibility of the reporter gene compression of the 3×FKBP-p65 activation domain fusion protein (−68 kDa). Therefore, if desired, clones may first be screened by western using anti-HA antibodies (see below). Clones expressing the highest levels of 3×FKBP-p65 should be selected for further analysis by transfection.

II. Stably Integrate the Target Plasmid

A. Transfect with plasmid vector or infect with retroviral vector containing the target gene under control of 12 ZFHD1-Binding Sites and a Minimal IL2 Promoter

If the LH-Z12-1-based plasmid vector is used, linearization with NotI or FspI will enhance the efficiency of integration.

B. Select Hygromycin-Resistant Clones

C. Screen for Clones with Low Background and High Dimerizer-Dependent Induction of Reporter Gene Expression

Transient Transfection Protocol

To screen clones it is convenient to transiently transfect cells in a 96-well format. Lipofectamine is used to introduce 50 ng total DNA/96-well under conditions recommended by the manufacturer (Gibco/BRL). If introducing both the transcription factor and target gene plasmids transiently, use 20 ng of each plasmid and 10 ng of carrier DNA (it may be necessary to optimize plasmid ratios and transfection conditions for each cell type). If only introducing one plasmid, bring to 50 ng with carrier DNA.

Following transfection, add medium +/−100 nM AP1510 dimerizer (or try a range of concentrations).

After overnight incubation (or longer), assay for target gene expression.

SEAP Assay Protocol

Secreted alkaline phosphatase activity can be easily measured from the supernatant of appropriately transfected cells using fluorescence-(see (10)), or chemiluminescence-(Tropix, Bedford, Mass.) based assays. Samples to be tested should first be incubated at 65° C. for 1 hour to inactivate endogenous alkaline phosphatase activity.

Western Protocol

The HA-epitope tagged transcription factors can be detected using commercially available anti-HA antibodies, including those from Babco (Richmond, Calif.; Cat. No. MMS101R-500). While the 3×FKBP-p65 activation domain fusion protein (˜68 kDa) should be easily detected, the DNA binding domain fusion, ZFHD1-3×FKBP (˜58 kDa), is expressed at lower levels and may not be visible.

References for Making Retrovirus

Helper-free retroviruses containing the target gene can be generated using the appropriate packaging vectors and cell lines as described elsewhere (9, 11).

Internal Ribosome Entry Sequence (IRES)

A tricistronic transcript expressing the transcription factor halves and the neo gene was created by inserting the ZFHD1-3×FKBP and neo genes downstream of the IRES from EMCV. To do this, 3 nucleotides, ACC, were added immediately 5′ to the 11th ATG of the EMCV IRES to create a Kozak consensus sequence and an NcoI site that encompasses the ATG. The ZFHD1-3×FKBP and neo genes were engineered to contain NcoI or compatible sites encompassing their ATGs which were then used to fuse the genes to the 11th ATG of the IRES. In the case of ZFHD1-3×FKBP, the amount of protein produced when it is the second cistron is only 10-20% of that produced when it is the first cistron. However, the relatively low level of expression of the DNA binding domain is still sufficient to direct high levels of induction of the target gene. Similarly, expression of the neo gene from the IRES is sufficient to confer resistance to G418.

Subcloning of the Transcription Factors

To put the expression of the transcription factors under control of an enhancer/promoter other than CMV, the coding region can be excised as a 4.94 kb EcoRI-BamHI fragment and subcloned. Note that this fragment must still be supplied with a poly(A) signal.

Alternatively, if the EcoRI site within the rabbit B-globin intron/poly A is mutagenized (see below), the CMV enhancer/promoter can be replaced as an EcoRI fragment.

A. Indirect Activation of Transcription

(1) General Description

The reagents described here can be used to induce protein dimerization. To do this, the protein(s) of interest is fused to one or more copies of human FKBP12, which can be dimerized by AP1510. AP1510 can be used, for example, to homodimerize a receptor in order to mimic authentic ligand-induced dimerization or to alter the intracellular localization of a protein by recruiting it to another protein anchored at a different location in the cell. Regulated dimerization of a number of proteins using related dimerizers has been described (1,10, 13-16)

The two plasmids included in this kit, pCF1E and pCMF2E, provide an assortment of components that can be easily manipulated to generate protein fusions whose activity and localization can be controlled by dimerizer.

(2) FKBP Expression Plasmids:

pCF1E

Inserts cloned into pCF1E as XbaI-SpeI fragments are transcribed under control of the human CMV enhancer promoter (C) and are expressed with a carboxy-terminal epitope tag (E, a 9 amino acid portion of the influenza hemagglutinin [HA gene). The XbaI-SpeI insert in pCF1E contains a single copy of FKBP12 (F1). The amino terminus of this fusion protein (upstream of the XbaI site) consists only of a methionine and an alanine. Thus, the localization of the fusion protein is determined by that which is fused to FKBP12, since FKBP12 alone will be localized predominantly to the cytoplasm.

pCMF2E

______ Inserts cloned into pCMF2E as XbaI-SpeI fragments are transcribed under control of the human CMV enhancer promoter (C) and are expressed with an amino-terminal myristoylation-targeting peptide (M) from the amino terminus of v-src and a carboxy-terminal epitope tag (E, a 9 amino acid portion of the influenza hemagglutinin [HA] gene). The myristoylation sequence directs the fusion protein to cellular membranes. The XbaI-SpeI insert in pCMF2E contains two tandem copies of FKBP12 (F2).

(3) General Information

Cloning Strategy

The basic strategy for creating protein fusions in this example is to amplify the coding sequence of interest so that it contains the six nucleotides specifying an XbaI site immediately 5′ to the first codon (beware not to create an overlapping Dam methylation sequence) and the six nucleotides specifying a SpeI site immediately 3′ to the last codon. Then, for example, to fuse the protein amino terminal to 2 FKBPs, clone the XbaI-SpeI fragment into the XbaI site of pCMF2E (XbaI and SpeI have compatible cohesive ends). If inserted in the proper orientation, the XbaI and SpeI sites, now flanking the new fusion protein, will be maintained, with the junction of the two peptides consisting of the two amino acids specified by the SpeI and XbaI sites that were fused. Or to fuse the XbaI-SpeI fragment carboxy-terminal to 2 FKBPs, insert it into the SpeI site of pCMF2E. In both cases, since the flanking XbaI and SpeI sites are maintained, additional fragments can still be fused at the amino- and carboxy-terminal ends.

This strategy can also be applied to create 3 tandem FKBPs. For example, the XbaI-SpeI fragment of pCF1E can be inserted into the SpeI site of pCMF2E (or vice versa).

If the sequence to be fused contains internal XbaI or SpeI sites, fusions can still be made either by using XbaI or SpeI at both ends, or by using NheI or AvrII which also generate ends that are compatible with XbaI and SpeI. Note, though, that in these cases the flanking XbaI and SpeI sites will not be regenerated.

The sequence between the SpeI and BamHI sites of both vectors encodes a carboxy-terminal HA epitope tag followed by a stop codon. Therefore, stop codons should not be included in the fused sequences.

Finally, XbaI-SpeI or XbaI-BamHI fragments can be cloned into either the pCM- or pC-vector backbones to create fusion proteins containing or lacking amino-terminal myristoylation-targeting peptides, respectively.

Targeting Fusions to the Nucleus

Replacement of the XbaI-SpeI insert in pCEN-F3p65/ZlF3/neo with an XbaI-SpeI fragment containing an FKBP fusion will generate a fusion protein containing an amino-terminal HA epitope tag and a nuclear localization signal from the SV40 large T antigen.

Production of Single Stranded DNA for Mutagenesis/Sequencing

pCEN vectors contain an M13 ori for rescue of the antisense strand. Oligonucleotides used for mutagenesis or sequencing should correspond to the sense stand of the vector.

C. Dimerizer

General Description

AP1510 is a synthetic dimerizer that can be used to induce homodimerization of FKBP12-containing fusion proteins. It is effective for both gene regulation applications and for general protein dimerization. AP1510 has no immunosuppressive activity and is non-toxic to cells.

AP1510 is conceptually related to FK1012, the prototype homodimerizer described in early dimerizer papers (10). Both molecules are symmetical homodimers of FKBP12 binding molecules. FK1012 is a semi-synthetic dimer of the natural product FK506. Positioning of the linker in the calcineurin binding domain of FK506 abolishes immunosuppressive activity while leaving FKBP12 binding unaffected. AP1510 is a smaller, simpler and completely synthetic molecule, in which two copies of an analog of the FK506 FKBP binding domain are directly linked.

AP1510 generally outperforms FK1012 in gene regulation and protein dimerization applications. In gene regulation applications, AP1510 activates transcription at lower concentrations and to a higher level than FK1012. In addition, AP1510 activates transcription efficiently in cells in which the transcription factor and reporter gene constructs are all stably integrated, whereas the activity of FK1012 is poor under these conditions. AP1510 has also been successfully used to dimerize a number of transmembrane receptors that are activated by oligomerization.

As AP1510 is a completely synthetic molecule, it readily supports modification and optimization for a given application. A variety of other synthetic dimerizing agents are disclosed in WO 96/06097 and WO 97/31898 for binding to FKBP-related domains.

General Information

Reconstituting AP1510

AP1510 (molecular weight 1190 Da) may be stored in lyophilized form. It should be reconstituted as a concentrated stock in an organic solvent. It is recommended that the lyophilized material be dissolved in absolute ethanol to make a 1 mM solution (eg. dissolve 250 mg AP1510 in 210 ml ethanol). After adding the appropriate volume of ice-cold ethanol, seal and vortex periodically over a period of a few minutes to dissolve the compound. Keep on ice during dissolution to minimize evaporation.

Storing and Handling AP1510

Once dissolved, the stock solution can be kept at −20° C. indefinitely, in a glass vial or an eppendorf tube. Further dilutions in ethanol can be similarly stored. At the bench, solutions in ethanol should always be kept on ice, and opened for as short a time as possible, to prevent evaporation and consequent changes in concentration.

Using AP1510

Working concentrations of AP1510 can be obtained by adding compound directly from ethanol stocks, or by diluting serially in culture medium just before use. In the latter case it is recommended that the highest concentration not exceed 5 uM, to ensure complete solubility in the (aqueous) medium. In either case, the final concentration of ethanol in the medium added to mammalian cells should be kept below 0.5% (a 200-fold dilution of a 100% ethanol solution) to prevent detrimental effects of the solvent on the cells.

Expected Results

In gene regulation applications, expression becomes detectable at an AP1510 concentration of approximately 10 nM, and peaks at approximately 100 nM. When SEAP or hGH reporter systems are used, expression can be easily detected after an overnight incubation with dimerizer. In our experience, the efficacy is generally similar for other dimerization applications. A range of concentrations from 1 nM to 1 uM will typically provide a good dose-response profile in both cases.

References for This Example

1. Belshaw, P. J., Ho, S. N., Crabtree, G. R., and Schreiber, S. L. Controlling protein association and subcellular localization with a synthetic ligand that induces heterodimerization of proteins. Proc. Natl. Acad. Sci 93, 4604-4607 (1996).

2. Ho, S. N., Biggar, S. R., Spencer, D. M., Schreiber, S. L., and Crabtree, G. R. Dimeric ligands define a role for transcription activation domains in reinitiation. Nature 382, 822-826 (1996).

3. Rivera, V. M., Clackson, T., Natesan, S., Pollock, R., Amara, J. F., Keenan, T., Magari, S. R., Phillips, T., Courage, N. L., Cerasoli Jr., F., Holt, D. A., and Gilman, M. A humanized system for pharmacologic control of gene expression. Nature Med. 2, 1028-1032 (1996).

4. Pomerantz, J. L., Sharp, P. A., and Pabo, C. O. Structure-based design of transcription factors. Science 267, 93-96 (1995).

5. Schmitz, M. L. and Baeurle, P. A. The p65 subunit is responsible for the strong transcription activating potential of NF-kB. EMBO J. 10, 3805-3817 (1991).

6. Standaert, R. F., Galat, A., Verdine, G. L., and Schreiber, S. L. Molecular cloning and overexpression of the human FK506-binding protein FKBP. Nature 346, 671-674 (1990).

7. Attar, R. M. and Gilman, M. Z. Expression cloning of a novel zinc-finger protein that binds to the c-fos serum response element. Mol. Cell. Biol. 12, 2432-2443 (1992).

8. Tanaka, M. and Herr, W. Differential transcriptional activation by Oct-1 and Oct-2: Interdependent activation domains induce Oct-2 phosphorylation. Cell 60, 375-386 (1990).

9. Morgenstern, J. P. and Land, H. Advanced mammalian gene transfer: high titre retroviral vectors with muplitple drug selection markers and a complementary helper-free packaging cell line. Nucl. Acids Res. 18, 3587-3596 (1990).

10. Spencer, D. M., Wandless, T. J., Schreiber, S. L., and Crabtree, G. R. Controlling signal transduction with synthetic ligands. Science 262, 1019-1024 (1993).

11. Pear, W. S., Nolan, G. P., Scott, M. L., and Baltimore, D. Production of high-titer helper-free retroviruses by transient transfection. Proc. Natl. Acad. Sci. USA 90, 8392-8396 (1993).

12. Rivera, V. M. and Gilman, M. Unpublished observations.

13. Luo, Z., Tzivion, G., Belshaw, P., Vavvas, D., Marshall, M., and Avruch, J. Oligomerization activates c-Raf-1 through a Ras-dependent mechanism. Nature 383, 181-184 (1996).

14. Holsinger, L. J., Spencer, D. M., Austin, D. J., Schreiber, S. L., and Crabtree, G. R. Signal transduction in T lymphocytes using a conditional allele of Sos. Proc. Natl. Acad. Sci. USA 92, 9810-9814 (1995).

15. Spencer, D. M., Graef, I., Austin, D. J., Schreiber, S. L., and Crabtree, G. R. A general strategy for producing conditional alleles of Src-like tyrosine kinases. Proc. Natl. Acad. Sci. USA 92, 9805-9809 (1995).

16. Spencer, D. M., Belshaw, P. J., Chen, L., Ho, S. N., Randazzo, F., Crabtree, G. R., and Schreiber, S. L. Functional anaysis of Fas signaling in vivo using synthetic inducers of dimerization. Curr. Biol. 6, 839-847 (1996).

### Example 2

### Cloning Angiostatin, Endostatin, Soluble Flk-1, Beta-Interferon, Angiopoietin-2 and 16 kD Prolactin for Regulated Expression Under Dimerizer Control

Target gene casettes for the regulated expression of angiostatin, endostatin, soluble Flk-1, beta-interferon, angiopoietin-2 and 16 kD prolactin may be prepared by analogy to constructs such as pZHWTx8SVSEAP and the corresponding target gene cassette used for regulated expression of hGH (see PCT/US96/09948). While the choice of DNA-binding domain and corresponding recognition sequence is left to the practitioner, the following experiments illustrate the use of the the ZFHD1 composite DNA-binding domain and its recognition sequence.

Constructs in which the expression of angiostatin, endostatin, soluble Flk-1, beta-interferon, angiopoietin-2 or 16 kD prolactin is placed under the control of a transcription factor utilizing the chimeric DNA binding domain ZFHD1 (Pomerantz et al., 1995) are prepared from the vector Z₁₂I-PL-2.

Z₁₂I-PL-2 Z₁₂]-PL-2 was constructed by replacing the HindIII-ClaI fragment from pZHWT×12-IL2-SEAP (Rivera et al 1996), in which expression of the SEAP reporter gene is driven by a basal promoter from a minimal IL-2 promoter (Rivera e tal., 1996) downstream from 12 tandem copies of a ZFHD1 binding site, with the oligonudeotides encoding the following polylinker:

```
5′-AAGCTTGCCCTGCAGCGGGAATTCCACTAGTCGAGATCTC
        HindIII PstI EcoRI SpeI BglII
CATCGAT-3′,
        ClaI
```

In addition, the ClaI-BamHI fragment, which contains the SV40 early gene intron and polyadenylation signal, was replaced by a ClaI-BamHI fragment that contains the 3′UTR from the SV40 late gene (amplified from pCAT3-Basic [Promega]). This target vector therefore contains, 5′ to 3′, 12 tandem copies of a ZFHD1 binding site, the basal promoter from the human IL2 gene, a polylinker, and a 3′UTR from the SV40 late gene.

In most embodiments it will be preferable to express the angiogenesis inhibitor genes or beta-interferon from constructs containing a cDNA rather than the complete gene including introns: for example, those cases in which the genes are to be introduced using a retroviral vector, or those cases in which the complete gene is especially large or the DNA coding capacity of the delivery vector limited. In these cases, cDNAs encoding the genes are amplified from mRNA from an appropriate human tissue source by RT-PCT using primers designed using the known mRNA sequences (Genbank accession numbers J00218 for beta-interferon, U01134 for soluble murine Flk-1, AF004327 for human angiopoietin-2, X54393 for human prolactin, AF018081 for endostatin (collage XVIII) and X05199 for angiostatin (plasminogen)). These genes are cloned as described below. Sites for restriction digestion are underlined.

Human Angiostatin:

(See Cao et al., J. Clin. Invest., 101:1055-1063, March 1998)

Amplify amino acids 1 to 472 from human plasminogen (secretory signal, the pre-activation peptide and kringle 1-4 region) using primers:

```
5′- t AAGCTT gccgccacc ATG GAA CAT AAG GAA GTG -3′
        HindIII
5′- t ATCGAT tta TTA ATC TGG AAG CAG GAC AAC -3′
              ClaI
```

Digest PCR product with HindIII-ClaI and ligate the resulting fragment into the HindIII-ClaI sites of the polylinker of Z₁₂I-PL-2. Confirm cloning by DNA sequencing. Human prolactin: (See Clapp et al., Endocrinology 133:1292-1299, 1993) The 16 kD fragment of human prolactin is cloned by amplifying amino acids 1 to 221 using primers:

```
5′- t AAGCTT gccgccacc ATG ATG AAA GGG TCC CTC
        HindIII
CTG -3′
5′- t ATCGAT tta TTA GCA GTT GTT GTT GTG GAT -3′
              ClaI
```

Clone HindIII-ClaI fragment into the polylinker of Z₁₂I-PL-2, as described for angiostatin.

Murine flk-1:

(see Lin et al. Cell Growth and Differentiation 9:49-58 1998) Amplify amino acids 1 to 736 using primers:

```
5′- t GAATTC gccgccacc ATG GAG AGC AAG GCG CTG -3′
            EcoRI
5′- t ATCGAT tta TTA CTG GCA GGT GTA GAG GCC -3′
              ClaI
```

Clone EcoRI-ClaI fragment into the polylinker of Z₁₂I-PL-2, as described above.

Human Angiopoietin-2:

(See Maisonpierre et al., Science 277:55-60, Jul. 4, 1997)

Amplify amino acids 1 to 497 using primers:

```
5′- t GAATTC gccgccacc ATG TGG CAG ATT GTT TTC -3′
              EcoRI
5′- t ATCGAT tta TTA GAA ATC TGC TGG TCG -3′
              ClaI
```

Clone EcoRI-ClaI fragment into the polylinker of Z₁₂I-PL-2, as described above.

Human B-Interferon:

Amplify amino acids 1 to 187 using primers:

```
5′- t GAATTC gccgccacc ATG ACC AAC AAG TGT CTC -3′
            EcoRI
5′- t ATCGAT tta TCA GTT TCG GAG GTA ACC -3′
              ClaI
```

Clone EcoRI-ClaI fragment into the polylinker of Z₁₂I-PL-2 as described above.

Human Endostatin:

(see Sasaki et al., EMBO J., 17:4249-4256, 1998)

Since human endostatin is a C-terminal fragment of collagen XVIII, in order to express it as a secreted protein, it must be fused to a signal sequence. For this purpose, the vector Z₁₂I-hGH-ss-XbaI was constructed by inserting a modified hGH cDNA between the EcoRI and ClaI sites of Z₁₂I-PL-2. The hGH cDNA was modified by mutagenesis to add an XbaI site immediately downstream of the secretion signal.

Amplify amino acids 1154 to 1337 from human collagen XVIII using primers:

```
5′- t TCTAGA CAC AGC CAC CGC GAC TTC -3′
                XbaI
5′- t ATCGAT tta CTA CTT GGA GGC AGT CAT -3′
                ClaI
```

Digest the PCR product with XbaI-ClaI, and ligate the resulting fragment into XbaI-ClaI-opened Z₁₂I-hGH-ss-XbaI. Perform DNA sequence to confirm cloning of the endostatin gene in frame with the secretory signal.

Equivalents

Those skilled in the art will recognize, or be able to ascertain using no more than routine experimentation, numerous equivalents to the specific materials and methods described herein. Such equivalents are considered to be within the scope of this invention.

1. A cell containing
(a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene encoding an angiogenesis inhibitor under the expression control of a transcriptional control element responsive to binding of ligand to the ligand binding domain.

2. The cell of claim 1 wherein the chimeric proteins multimerize upon addition of ligand and wherein transcription of the target gene is responsive to the multimerization of the chimeric proteins.

3. The cell of claim 1 wherein the ligand binding domain is selected from the group consisting of an immunophilin domain, a cyclophilin domain, a steroid hormone binding domain and an antibiotic binding domain.

4. The cell of claim 1 wherein the angiogenesis inhibitor is selected from the group consisting of thrombospondin, angiostatin, endostatin, angiostatin-endostatin fusion proteins, angiopoietin-2, a soluble receptor for VEGF, a dominant negative form of VEGF, anti-VEGF antibodies, soluble Tie2/Tek receptor and the 16 kD fragment of prolactin.

5. The engineered cell of claim 1 or 4 in which the target gene encodes a peptide sequence of human origin.

6. A cell containing
(a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene encoding a tumor specific antigen under the expression control of a transcriptional control element responsive to binding of ligand to the ligand binding domain.

7. The cell of claim 6 wherein the chimeric proteins multimerize upon addition of ligand and wherein transcription of the target gene is responsive to the multimerization of the chimeric proteins.

8. The cell of claim 6 wherein the ligand binding domain is selected from the group consisting of an immunophilin domain, a cyclophilin domain, a steroid hormone binding domain and an antibiotic binding domain.

9. A cell containing
(a) a DNA construct encoding a chimeric protein consisting essentially of (i) a receptor domain capable of binding to a selected ligand, (ii) a transcription activation domain, heterologous with respect to the receptor domain, (iii) and a DNA binding domain; and
(b) a target gene encoding beta-interferon or a cytokine under the expression control of a transcriptional control element responsive to binding of ligand to the ligand binding domain.

10. The cell of claim 9 wherein the ligand binding domain is selected from the group consisting of a steroid hormone binding domain and an antibiotic binding domain.

11. The cell of claim 6 or 9 in which the target gene encodes a peptide sequence of human origin.

12. A recombinant virus containing
(a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene encoding an angiogenesis inhibitor, a tumor specific antigen, a cytokine or beta-interferon under the expression control of a transcriptional control element responsive to binding of ligand to the ligand binding domain.

13. The recombinant virus of claim 12 wherein the virus is selected from the group consisting of adenovirus, adeno-associated virus, retrovirus and herpesvirus.

14. A method for rendering cells capable of regulatable expression of a target gene following exposure of the cells to a selected ligand, which method comprises introducing into the cells:
(a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene under the expression control of a transcriptional control element responsive to binding of ligand to the ligand binding domain,
wherein the target gene encodes an angiogenesis inhibitor or a tumor specific antigen.

15. The method of claim 14 wherein the angiogenesis inhibitor is selected from the group consisting of thrombospondin, angiostatin, endostatin, angiostatin-endostatin fusion proteins, angiopoietin-2, a soluble receptor for VEGF, a dominant negative form of VEGF, anti-VEGF antibodies, soluble Tie2/Tek receptor and the 16 kD fragment of prolactin.

16. A method for rendering cells capable of regulatable expression of a target gene following exposure of the cells to a selected ligand, which method comprises introducing into the cells:
(a) a DNA construct encoding a chimeric protein consisting essentially of (i) a receptor domain capable of binding to a selected ligand, (ii) a transcription activation domain, heterologous with respect to the receptor domain, (iii) and a DNA binding domain; and
(b) a target gene encoding beta-interferon or a cytokine under the expression control of a transcriptional control element responsive to binding of ligand to the ligand binding domain.

17. The method of claim 14 or 16 wherein the DNA constructs are introduced into cells maintained in vitro.

18. The method of claim 14 or 16 wherein the DNA constructs are introduced into cells present within a host organism.

19. The method of claim 14 wherein the chimeric proteins multimerize upon addition of ligand and wherein transcription of the target gene is responsive to the multimerization of the chimeric proteins.

20. The method of claim 14 or 16 wherein the ligand binding domain is selected from the group consisting of an immunophilin domain, a cyclophilin domain, a steroid hormone binding domain and an antibiotic binding domain.

21. A method for treating cancer in a mammalian host organism containing cells which:
contain (a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene under the expression control of a transcriptional control element responsive to binding of ligand to the ligand binding domain; and
which express the target gene, following exposure to the selected ligand;
wherein the target gene encodes an angiogenesis inhibitor, a tumor-specific antigen or a cytokine;
which method comprises administering to said mammalian host an effective amount of a selected ligand capable of binding to the chimeric protein to effect observable expression of the target gene.

22. A method for treating MS episodes in a mammalian host organism containing cells which:
contain (a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene encoding beta-interferon under the expression control of a transcriptional control element responsive to binding of ligand to the ligand binding domain; and
which express the target gene, following exposure to the selected ligand;
which method comprises administering to said mammalian host an effective amount of a selected ligand capable of binding to the chimeric protein to effect observable expression of the target beta-interferon gene.

23. A method for treating HIV infection in a mammalian host organism containing cells which:
contain (a) a first DNA construct or pair of first DNA constructs encoding chimeric proteins comprising (i) at least one receptor domain capable of binding to a selected ligand and (ii) another protein domain, heterologous with respect to the receptor domain, and
(b) a target gene under the expression control of a transcriptional control element responsive to binding of ligand to the ligand binding domain; and
which express the target gene, following exposure to the selected ligand;
wherein the target gene encodes a ribozyme or antisense message directed against an HIV nucleotide sequence;
which method comprises administering to said mammalian host an effective amount of a selected ligand capable of binding to the chimeric protein to effect observable expression of the target gene.
